(19) United States
(12) Patent Application Publication
(10) Pub. No.: US 2022/0110779 A1
(43) Pub. Date: Apr. 14, 2022

(54) PORTABLE URINAL DEVICE

(71) Applicant: Gaylene Ricketts, Yukon, OK (US)

(72) Inventor: Gaylene Ricketts, Yukon, OK (US)

(21) Appl. No.: 17/070,105

(22) Filed: Oct. 14, 2020

(51) Int. Cl. A61F 5/451

(52) U.S. Cl. CPC A61F 5/451; A61F 2005/4402

(57) ABSTRACT

The disclosure comprises a portable urinal device. The urinal device comprises a housing with an exterior surface and defines an interior cavity along with a first actuator and a second actuator. The urinal device also comprises a waste collector assembly. The waste collector assembly includes a bracket arm, activated by the first actuator from a closed position. The waste collector assembly further includes a plurality waste collector brackets. The waste collector brackets are configured to extend from the housing and translate vertically in the interior cavity. The translation of the waste collector brackets is activated by the second actuator. The waste collector assembly also includes a waste collector. The waste collector is configured to stay in a folded configuration while the waste collector assembly is in the housing. The waste collector is configured to expand to a container configuration when the waste collector bracket is extended from the interior cavity.

### TECHNICAL FIELD

The present disclosure relates generally to the field urinal devices, and more particularly, to systems and methods using a portable urinal device and managing disposal of the urinal device.

### BACKGROUND OF THE INVENTION

Urination is a necessary bodily function to relieve the body of wastes. Unfortunately, timing and accessibility of suitable facilities can be factors that can make the need to urinate extremely uncomfortable or inconvenient. Thus, there exists a need for a solution that addresses the ability to access a urinal. For all of these reasons, portable elimination devices offer an alternative solution.

### SUMMARY OF THE INVENTION

The present disclosure meets the needs described above comprising a method and system for a portable urinal. The portable urinal device can comprise a housing. The housing can comprise an exterior surface that defines an interior cavity. The urinal device can also comprise a waste collector assembly. The waste collector assembly can comprise a bracket arm. The waste collector assembly can further comprise a waste collector bracket configured to be removably coupled from the bracket arm. The bracket arm can be configured to extend from the housing. The waste collector assembly can further comprise a waste collector. The waste collector can be coupled to the waste collector bracket. The waste collector can be configured to stay in a folded configuration while the waste collector assembly is in the housing and configured to expand a container configuration when the waste collector bracket extends from the housing.

In another embodiment, a urinal device can comprise a housing with an exterior surface. The housing can also define an interior cavity. The urinal device can include a first actuator and a second actuator. Both actuators can be oriented on the housing. The second actuator can comprise at least one spring loaded member. The urinal device can also comprise a waste collector assembly. The waste collector assembly can comprise a bracket arm. The bracket arm can be activated by the first actuator from the closed configuration. The waste collector assembly can further include a plurality of waste collector brackets. Each waste collector bracket can be configured to extend from the housing when coupled to the bracket arm. The waste collector brackets can be arranged in a stackable cartridge. The individual waste collector brackets of the cartridge can translate vertically in the interior cavity. The translation of the packet can be activated by the second actuator. The spring loaded member of the second actuator can provide stationary support to at least one of a plurality of waste collector brackets. A waste collector coupled to the waste collector bracket can be configured to stay in a folded configuration while the waste collector assembly is in the housing. The waste collector can be configured to expand to a container configuration when the waste collector bracket is extended from the interior cavity.

In another aspect, a method for using the urinal device can comprise activating the urinal device from a closed configuration from an open configuration. Activating the urinal device to the open configuration can comprises extending a waste collector assembly from an interior cavity of the urinal device. The method can include providing a disposable substance to the waste collector assembly. The method can further comprise detaching a portion of the waste collector assembly for disposal.

In view of the foregoing, it will be appreciated that the present disclosure provides a system and method for using a portable urinal. The specific systems and techniques for accomplishing the advantages described above will become apparent from the following detailed description of the embodiments and the appended drawings and claims.

### BRIEF DESCRIPTION OF THE DRAWINGS

FIG. 1 depicts an isometric view of an embodiment in a closed configuration.

FIG. 2 depicts an isometric view of the embodiment in FIG. 1 in an open configuration.

FIG. 3 depicts an isometric view of the embodiment in FIG. 1 in an open configuration along with the waste collector in a container configuration.

FIG. 4 depicts a partial isometric top view of the embodiment in FIG. 1 with a top portion of the housing removed.

FIG. 5 depicts a partial isometric bottom view of the embodiment in FIG. 1 with a bottom portion of the housing removed.

FIG. 6 depicts a partial isometric top view of a second embodiment with a top portion of the housing removed.

FIG. 7 depicts a partial isometric top view of the embodiment in FIG. 6 with a top portion of the housing removed.

FIG. 8 depicts a rear cross-sectional view isometric view of the embodiment in FIG. 6.

FIG. 9 depicts an isometric view of a third embodiment in a closed configuration.

FIG. 10 depicts an isometric view of the embodiment in FIG. 9 in an open configuration

FIG. 11 depicts a partial isometric top view of the embodiment in FIG. 9 with a top portion of the housing removed.

FIG. 12 depicts a partial isometric side view of the embodiment in FIG. 9 with a top portion of the housing removed.

FIG. 13 depicts an isometric top view of the embodiment in FIG. 9 with a top portion of the housing removed.

FIG. 14 depicts an isometric bottom view of the embodiment in FIG. 9 with a bottom portion of the housing removed.

FIG. 15 depicts an isometric top view of a fourth embodiment with a top portion of the housing removed.

FIG. 16 depicts an isometric view of the embodiment in FIG. 9 in an open configuration with a portion of the top cover of the housing removed.

### DETAILED DESCRIPTION

Illustrative embodiments of the disclosure will now be described more fully hereinafter with reference to the accompanying drawings, in which some, but not all embodiments of the disclosure are shown. The disclosure may be embodied in many different forms and should not be construed as limited to the embodiments set forth herein; rather, these embodiments are provided so that this disclosure will satisfy applicable legal requirements.

Whenever appropriate, terms used in the singular also will include the plural and vice versa. The use of “a” herein means “one or more” unless stated otherwise or where the use of “one or more” is clearly inappropriate. The use of “or” means “and/or” unless stated otherwise. The use of “comprise,” “comprises,” “comprising,” “include,” “includes,” and “including” are interchangeable and not intended to be limiting. The term “such as” also is not intended to be limiting. For example, the term “including” shall mean “including, but not limited to.”

The following description is provided as an enabling teaching of the disclosed articles, systems, and methods in their best, currently known embodiments. To this end, those skilled in the relevant art will recognize and appreciate that many changes can be made to the various aspects of the articles, systems, and methods described herein, while still obtaining the beneficial results of the disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

As used throughout, the singular forms “a,” “an” and “the” include plural referents unless the context clearly dictates otherwise. Thus, for example, reference to “a gasket” can include two or more such gaskets unless the context indicates otherwise.

As used throughout, “substantially” with respect to a measure can refer to a range of values comprising +/−10 degrees. For example, substantially orthogonal, normal, or parallel can include embodiments, where the referenced components are oriented +/−10 degrees of being classified as orthogonal, normal, or parallel respectively.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

The word “or” as used herein means any one member of a particular list and also includes any combination of members of that list.

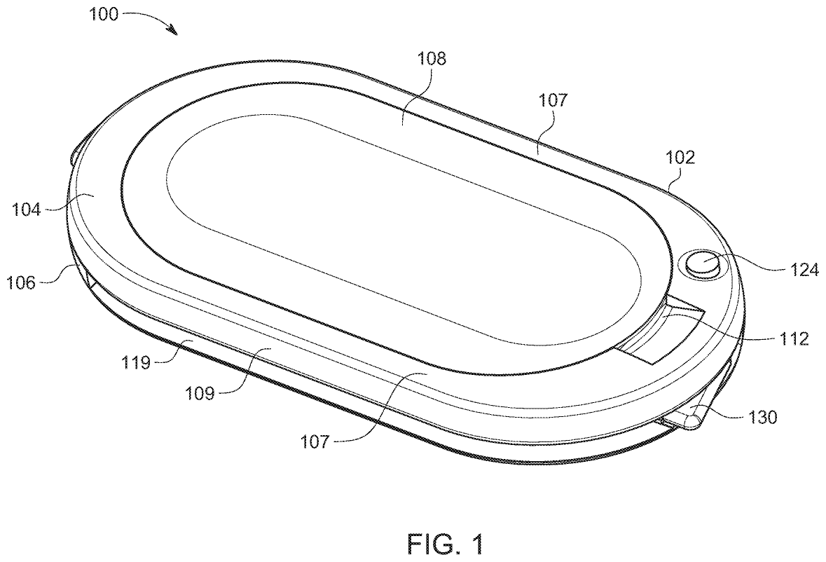

As shown in FIG. 1 the portable urinal system can be a urinal device 100. The urinal device 100 can comprise a housing 102. The housing 102 can comprise an exterior surface 108 that defines and interior cavity 103 (not shown). In a further aspect, the housing 102 can comprise a top portion 104 that mates with a bottom portion 106. The housing can also comprise a side surface 109. The side surface 109 can define a slit aperture 119 that serves as a passage for a waste collector assembly 114 (not shown) to enter and exit the interior cavity of the housing 102. The housing 102 can be comprised of a metallic material. In another embodiment, the housing 102 can be comprised of rigid and/or semi rigid material such as a plastic. In another aspect, the exterior surface 108 can include gripping features and/or textured patterns 107. These gripping features can increase the tactile grip, which can reduce the urinal device 100 from slipping out of the user's hand in the event that the urinal device 100 gets wet. In a further aspect, the textured patterns can be etched patterns on the exterior surface 108. The gripping features can be portions of rubber or silicone adhered to the exterior surface to reduce slippage.

In a further aspect, the exterior surface 108 can comprise a top cover 110. The top cover 110 can be used to access the interior cavity 103 of the housing 102. In another aspect, the exterior surface 108 can define an access divot 112. The access divot 112 can be an indentation in the exterior surface 108. The access divot can be in proximity to the top cover 110. The access divot 112 can provide a user with additional dexterity in using their finger or tool to open the top cover 110 for gaining access to the interior cavity 103. In another embodiment, the access divot 112 can be defined in the top cover 110. The top cover can also 110 can be made of transparent or semi-transparent material to permit the user to view into the interior cavity 103 to identify the contents of the interior cavity.

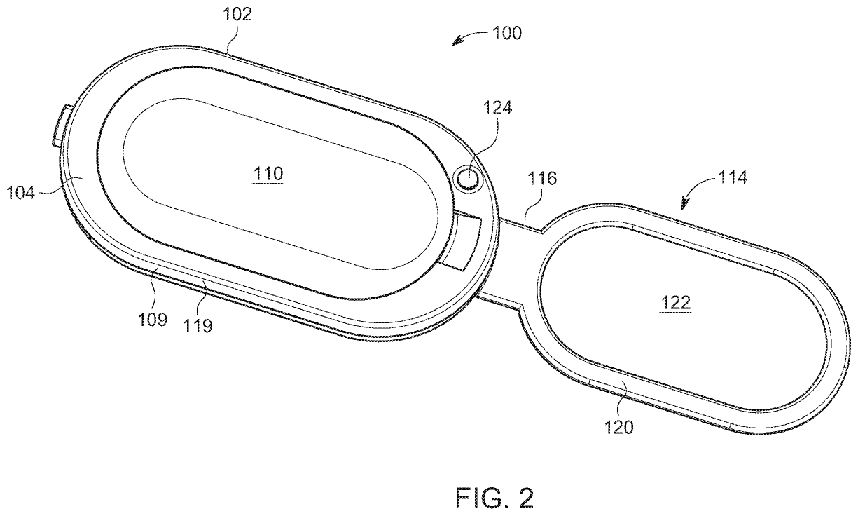

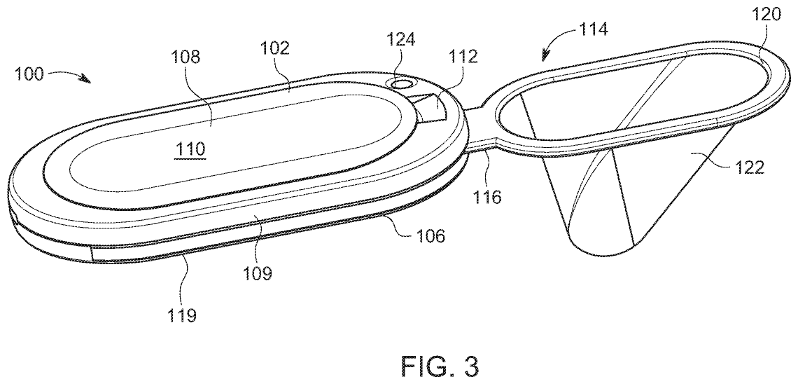

As shown in FIG. 2, the urinal device 100 can transition from the closed configuration in FIG. 1 to the open configuration in FIG. 2. In the open configuration, the waste collector assembly 114 extends from the interior cavity of the housing 102. In the closed configuration the waste collector assembly 114 resides in the interior cavity 103 of the housing 102. In FIG. 3, the waste collector 122 of the waste collector assembly 114 can transition from a folded configuration to a container configuration for holding the fluids and/or substances. While in the folded configuration, the waste collector 122 can have approximately the same dimensions of the waste collector bracket 122 to facilitate storage of the waste collector assembly 114 in the interior cavity 103.

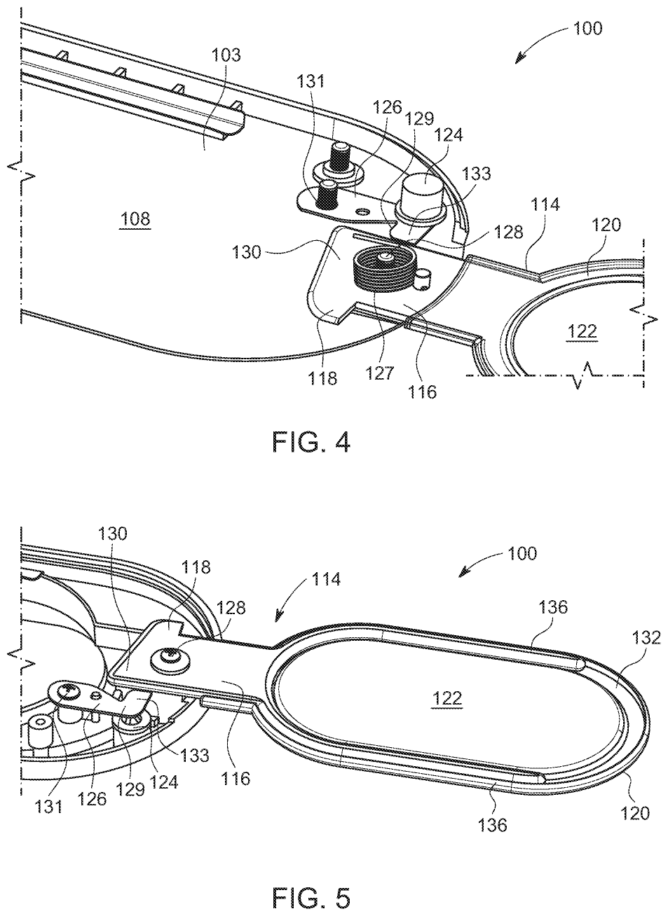

As shown in FIG. 4, the waste collector assembly 114 can comprise a bracket arm 116 coupled to the waste collector bracket 120. The bracket arm 116 can comprise a member that is coupled to the bottom portion 106 of the housing 102. In another embodiment, the bracket arm 116 can be coupled to the top portion 104 (not shown). The bracket arm 116 can be configured to rotate around a pivot point 128. The bracket arm 116 can rotate around the pivot point 128 as the bracket arm 116 transitions the urinal device 100 from the open configuration the closed configuration. Similarly, the bracket arm 116 can rotate around the pivot arm as the urinal device transitions from the open configuration to the closed configuration. In a further aspect, the bracket arm 116 can be spring loaded with a waste collector assembly spring 127. The waste collector assembly spring 127 can be a rotational spring with one end anchored to a portion of an interior surface of housing 102 and a second end of the rotational spring anchored to a surface of the bracket arm 116.

The bracket arm 116 can be activated to transition from a closed configuration to an open configuration with the use of a waste collector assembly actuator 124. The waste collector assembly actuator 124 can comprise a button or a cylindrical shaft. The button 124 can be activated when the user applies a normal force relative to the housing by pressing the button. The button can be oriented to engage a free end 133 of the actuator hook member 126. The actuator hook member 126 can comprise a cantilevered member comprising a fixed end at fastener 131 and the free end 133. The free end 133 can further comprise a hook portion 129. In the closed configuration, the hook portion 129 at the free end 133 can be oriented to engage with a grip portion 118 of the bracket arm 116. The grip portion is on a region of the bracket arm that extends away from the surface of the bracket arm to serve as an engagement point. To transition from the closed configuration to the open configuration, the button of the actuator 124 can be pressed, which can force the free end 133 to flex vertically. The resultant action can cause the hook portion 129 of the hook member 126 to disengage the grip portion 118 of the bracket arm 116. When the engagement between the hook portion 129 and the grip portion 118 occurs, the bracket arm 116 can freely rotate to extend the waste collector assembly 114 away from the interior cavity 103 of the housing 102, completing the transition to the open configuration. As discussed earlier, the urinal device 100 can include a rotational spring 127. The transition from the closed configuration to the open configuration can be expedited by an embodiment with the waste collector assembly spring 127. In this aspect, the waste collector assembly spring 127 can be biased such that the potential energy is in stored when the urinal device 100 is in the closed configuration. When the actuator 124 is activated to transition the urinal device from the closed configuration to the open configuration, the potential energy stored in the waste collector assembly spring is released causing the bracket arm 116 to rotate swiftly from the interior cavity 103. In a further aspect, the bracket arm 116 can be comprised of a rigid or semi-rigid material such as a plastic or metal.

As shown in FIG. 5, the waste collector assembly 114 further comprises a waste collector bracket 120. The waste collector bracket 120 can comprise a bottom surface that is configured in a channel configuration 132. The shape of the channel configuration 132 can be used as a fixture to provide engagement between the waste collector bracket 120 and the bracket arm 116. In a further aspect, the bracket arm 116 can have extender members 136. The extender members 136 can be shafts that protrude from a distal portion of the bracket arm 116. The extender members 136 can be used as an engagement surface between the bracket arm 116 and the waste collector bracket 120. The waste collector bracket 120 can engage the bracket arm 116 when the extender members 136 rest in the channel 132. In a further aspect, the channel 132 of the waste collector bracket 120 can be comprised of a rigid or semi-rigid material such as a plastic or metal. Other embodiments of the waste collector bracket 120 can comprise combinations of polymers, rubber, or silicone. In a further aspect, the waste collector bracket 120 can be comprised of a corrugated cardboard to preserve some rigidity while providing increased biodegradability of the waste collector bracket for disposal. Similarly, the waste collector 122 can be comprised of a biodegradable plastic, or wax-lined paper.

As shown in FIGS. 4 and 5, in a further aspect, the bracket arm 116 can further comprise an extender wedge 130. The extender wedge 130 can be a protrusion that extends from the surface of the bracket arm 116. When the user attempt to transition the waste collector assembly 114 from the open configuration to the closed configuration, the user can initially begin the rotation of the bracket arm 116 by applying a force to the extender members 136. After the extender arms 136 have been returned to the interior cavity 103, the extender wedge 130 serves as a grip to allow the user to continually apply a force to the bracket arm, until the waste collector assembly 114 has been re-engaged into the closed position.

The waste collector bracket 120 can be coupled to a waste collector 122. The waste collector 122 can be configured to be folded into the waste collector assembly 114 when the waste collector assembly 114 is in the interior cavity (closed configuration). When the waste collector assembly 114 is extended into the open configuration, the user has access to the waste collector 122 of the waste collector assembly 114. The user can then provide fluids or substances into the waste collector for their desired purpose (i.e. urination or scooping pet wastes). The waste collector 122 can be comprised of a plastic, polymer, rubber or silicone. The weight liquids and/or substances can cause the waste collector 122 to transition from a folded configuration to a container configuration. In other aspects, the user can aid the transition by manually pressing the waste collector surface in the folded configuration to open the waste collector. When the user completes using the urinal device 100, the user can disengage the waste collector bracket 120 (including the waste collector 122) from the bracket arm 116. The user can then discard the waste collector 122 and waste collector bracket 120. In one aspect, the waste collector bracket 120 is removably coupled to the bracket arm 116. The removably coupled configuration permits the user to disengage the waste collector bracket 120 from the extender arms 136. In an alternate embodiment, the waste collector assembly 114 can be configured such that the only removable/discarded portion would be the waste collector 122. For example, after the waste collector has been filled with removable substances either liquid or solid, only the waste collector 122 is removed from the waste collector assembly 114.

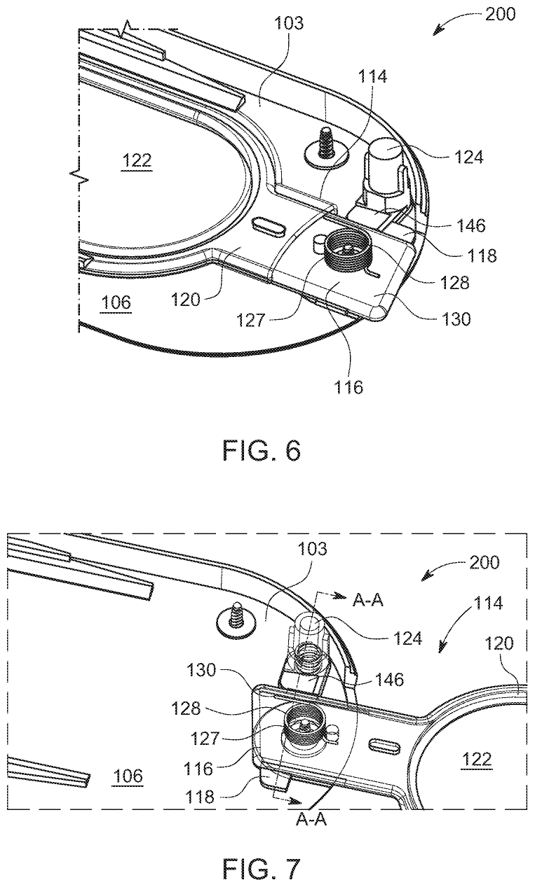

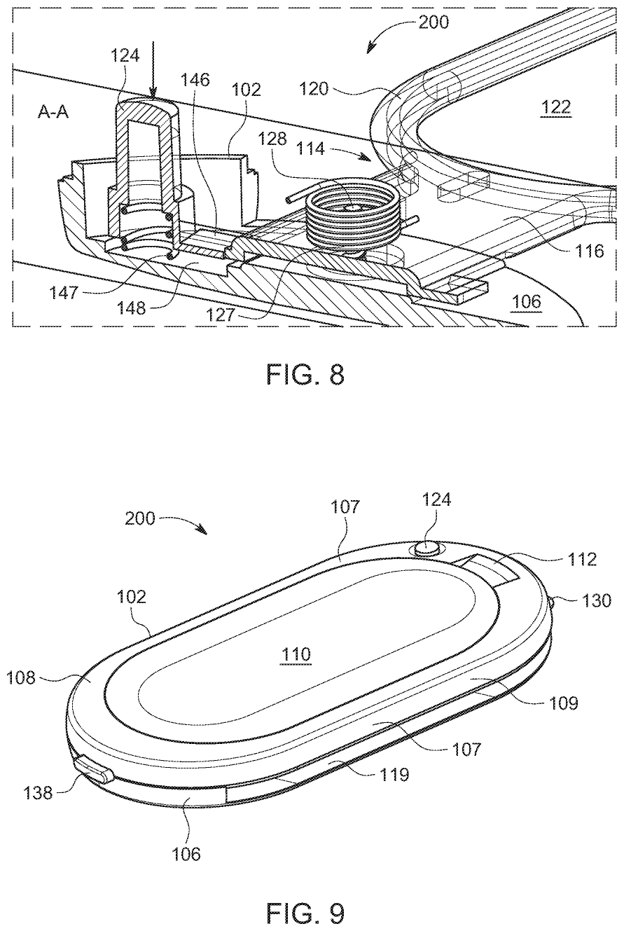

As, shown in FIGS. 6, 7, and 8, urinal device 200 can comprise another embodiment for the waste collector assembly actuator 124. The waste collector assembly actuator 124 can comprise a button or a cylindrical shaft. Towards the base of the cylindrical shaft 124 can be a flange 146 that extends from the base. As shown in FIG. 6, urinal device 200 is in the closed configuration. In the closed configuration, the flange 146 is engaged to grip portion 118 of the bracket arm 116, which prevents the bracket arm from rotating around the pivot point 128. As shown, inn FIGS. 7 and 8, urinal device 200 is in an open configuration. To transition to the open configuration, a user can apply a normal force to the waste collector assembly actuator 124. As shown in FIG. 8, the waste collector assembly actuator 124 can comprise a compression spring 147. When the normal force is applied, the waste collector assembly actuator 124 can be lowered into the recess 148. The recess 148 can be a divot in the bottom portion 106 of the housing interior. When waste collector assembly actuator 124 is lowered into the recess 148, the flange 146 can no longer engage the grip portion 118 of the bracket arm 116. The bracket arm 116 can then be free to rotate around the pivot point 128. When the actuator 124 is no longer pressed, the potential energy stored in the compression spring 147 can be released, which elevates the waste collector assembly actuator 124 from the recess 148 back to a resting position.

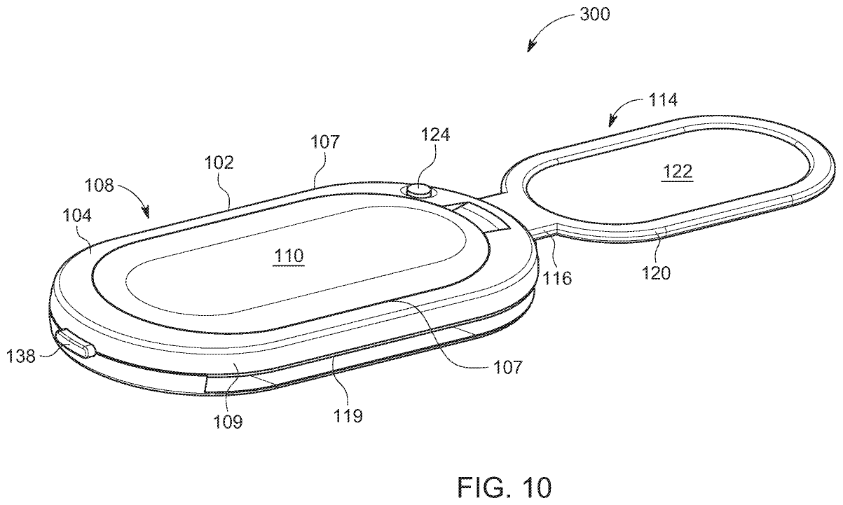

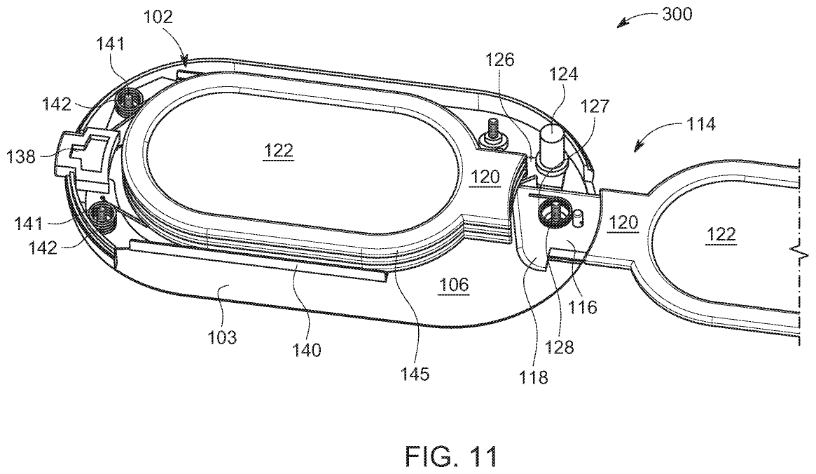

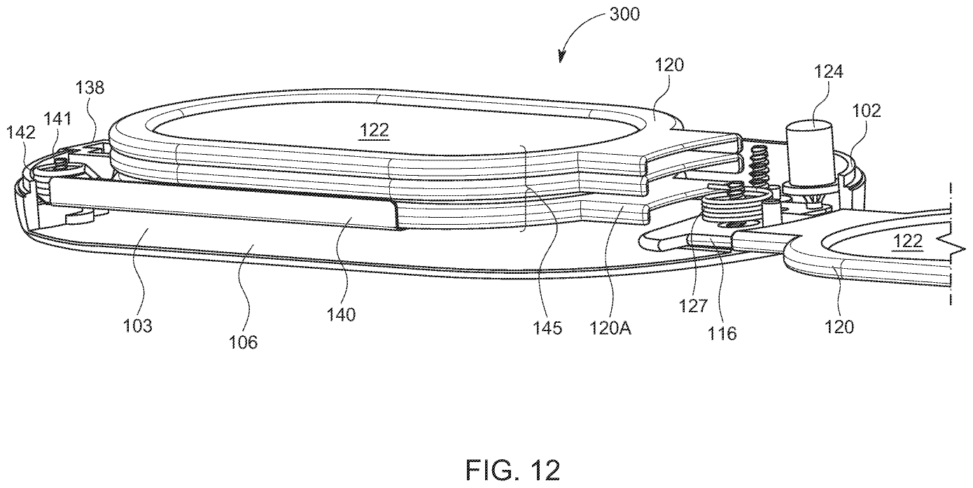

In FIG. 9 and FIG. 10, the urinal device 300 can comprise multiple removable waste collector brackets 120. The urinal device 300 shown in the closed configuration in FIG. 9 and open configuration in FIG. 10 both further comprise a second actuator, the bracket release actuator 138. As shown in FIG. 11 and FIG. 12, multiple waste collector brackets 120 can be stacked on top of each other to form a stack or cartridge 145. The cartridge of waste collector brackets 120 with associated waste collectors 122 can be a method for packaging multiple waste collector brackets and waste collectors, which also facilitates ease of refilling the urinal device 300. In order to transition between the multiple waste collector brackets in a stack, urinal device 300 can utilize the bracket release actuator 138.

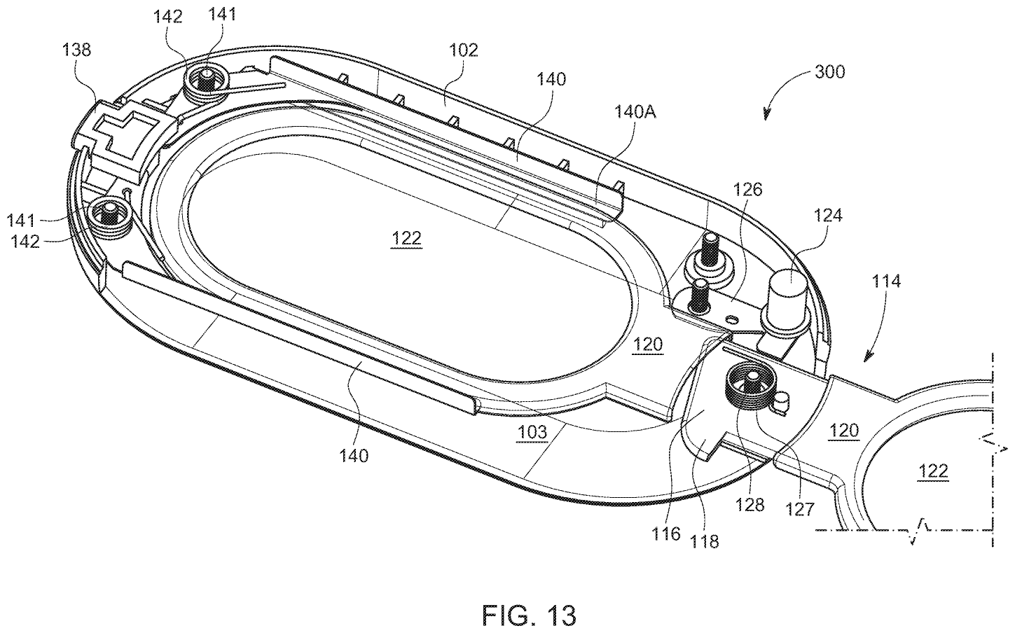

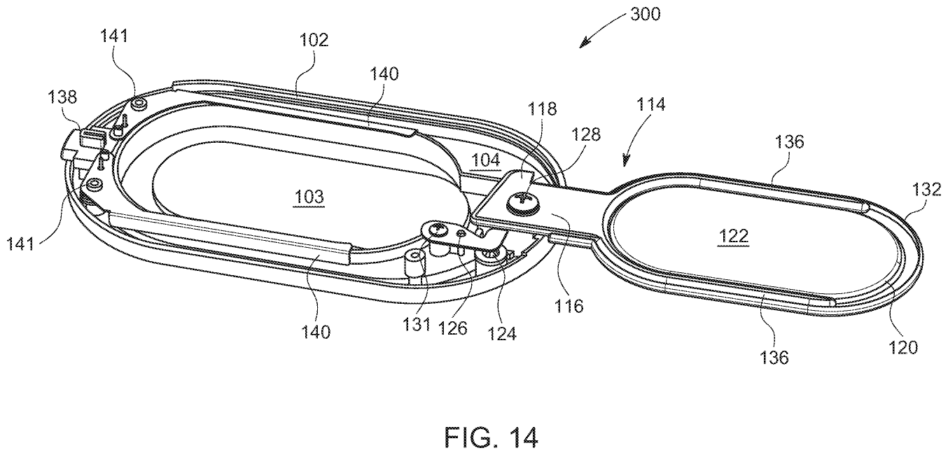

As shown in FIG. 13 and FIG. 14, the bracket release actuator 138 can be a button that when pressed activates the movement of release members 140. The release members 140 can be a set of shafts or rods coupled to the bracket release actuator 138. The release members 140 can be used to engage the stack of waste collector brackets 120. When the bracket release actuator 138 is pressed, the coupling between the release members 140 can cause and end of the release members to rotate outward away from the center of the waste collector brackets 120. The release member 140 can rotate around the auxiliary pivot points 141. As the release members 140 rotate around their respective auxiliary pivot points 141, the release members 140 can release or disengage from the waste collector bracket 120. As the release members 140 disengage from the waste collector bracket 120, the cartridge 145(stack) of waste collector brackets can translate vertically from a stationary position in an upper region of the interior cavity 103 to the bottom portion 106 of the housing 102. The lowest waste collector bracket 120A can then engage the bracket arm 116 as the channel 132 of the lowest waste collector bracket 120A comes to rest on the extender members 136 of the bracket arm. In a further aspect, the release members 140 can be coupled to release springs 142. The release springs 142 can be biased to hold the release members 140 inward to engage the waste collector bracket 120/cartridge 145 of waste collector brackets, holding the waste collector bracket 120/cartridge 145 in a stationary position. The release members 140 can have a surface 140A configured to engage the waste collector bracket 120.

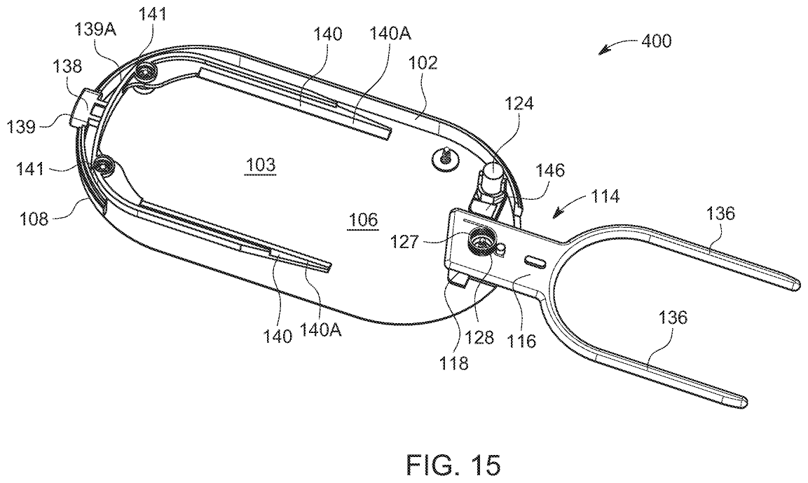

As shown in FIG. 15, the urinal device 400 differs from the urinal device 300 in that the bracket release actuator 138 in urinal device 400 can comprise a unitary piece. The unitary formation of the bracket release actuator 138 can comprise subcomponents. In urinal device 400, the subcomponents of the bracket release actuator 138 can comprise a protrusion 139, a crossbeam member 139A, and release members 140. The protrusion 139 can extend from the exterior surface 108 to serve as point where is applied. Similar to urinal device 300, the release members 140 can have a surface 140A configured to engage the waste collector bracket 120. As a unitary piece, the bracket release actuator 138 will flex when the protrusion 139 is pressed inward towards the interior cavity 103 of the housing 102. The protrusion 139 is coupled to a crossbeam member 139A; the crossbeam member 139A is connected to both of the release members 140. The deflection of the crossbeam member 139A can cause the release members 140 to rotate around the auxiliary pivot points 141. The rotating release members 140 can result in either engaging or disengaging the waste collector bracket 120(not shown) from a stationary position. In one aspect, the release actuator can be comprised of a thin metal to permit the necessary flexion and in another aspect the release actuator can comprise a flexible plastic.

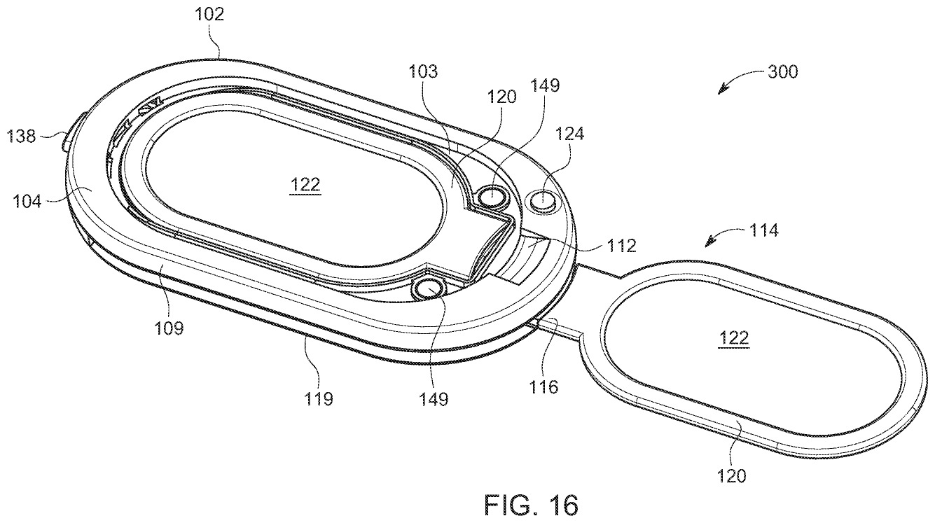

As shown in FIG. 16, top cover 110 can be removed to provide access to the multiple waste collector brackets 120 in the interior cavity 103. In one aspect, a portion of the top cover 110 (not shown) can comprise metallic components and/or a magnet. The metallic components and/or a magnet components of the top cover 110 can be oriented to mate with the housing magnets 149 oriented in the top portion 104 of the housing. The magnetic engagement between the top cover 110 and top region of the housing 104 can secure a seal between the top cover 110 and interior cavity 103. The user can still disengage the top cover 110 via the access divot 112. In another aspect, the top cover and the top portion 104 can be secured with a snap fit engagement. In a further aspect, a portion of the top cover 110 can comprise transparent or semi-transparent material, which would allow the user to identify how many waste collector brackets are left.

While the disclosure has been described in connection with what is presently considered to be the most practical and various embodiments, it is to be understood that the disclosure is not to be limited to the disclosed embodiments, but on the contrary, is intended to cover various modifications and equivalent arrangements comprised within the spirit and scope of the appended claims.

What is claimed is:

1. A urinal device comprising:
a housing comprising an exterior surface that defines an interior cavity; and
a waste collector assembly comprising:
a bracket arm,
a waste collector bracket configured to extend from the housing and removably coupled to the bracket arm, and
a waste collector coupled to a waste collector bracket configured to stay in folded configuration while the waste collector assembly is in the housing and configured to expand when the waste collector bracket is extended from the housing.

2. The urinal device of claim 1, wherein the exterior surface defines an opening for providing access to the interior cavity.

3. The urinal device of claim 1, further comprising an actuator oriented on the exterior surface of the housing, the actuator being configured to activate the bracket arm from a stationary position.

4. The urinal device of claim 3, wherein the bracket arm is spring-loaded.

5. The urinal device of claim 3, wherein the actuator comprises hook member, the hook member configured to engage a grip portion of the waste collector assembly.

6. The urinal device of claim 1, wherein the waste collector assembly is configured to rotate around a pivot point as the waste collector assembly extends from the housing in to an open configuration, and the waste collector assembly is configured to return to a closed configuration by rotating around a pivot point.

7. The urinal device of claim 1, wherein the bracket arm further comprises an extender wedge located on a surface of the bracket arm configured to facilitate returning the waste collector assembly from an open configuration to a closed configuration.

8. The urinal device of claim 1, wherein the housing comprises a top portion and bottom portion.

9. The urinal device of claim 1, wherein the bracket arm comprises two extender members.

10. The urinal device of claim 1, wherein a bottom surface of the waste collector bracket defines a channel, the channel being configured to engage a top surface of the bracket arm.

11. A urinal device comprising:
a housing comprising an exterior surface that defines an interior cavity;
a first actuator oriented on the exterior surface of the housing;
a second actuator oriented on the exterior surface of the housing comprising at least one member; and
a waste collector assembly comprising:
a bracket arm, wherein the first actuator being configured to activate the bracket arm from a stationary position,
a plurality of waste collector brackets configured to extend from the housing based on activation of the first actuator, wherein the plurality of waste collector brackets translates vertically in the interior cavity based on activation by the second actuator, and wherein the at least one member provides stationary support to at least one of the plurality of waste collector brackets, and
a plurality of waste collectors coupled individually to a waste collector bracket of the plurality of waste collector brackets and configured to stay in a folded configuration while the waste collector assembly is in the housing and configured to expand to a container configuration while the least one of the plurality of waste collector brackets is extended from the interior cavity.

12. The urinal device of claim 11, wherein the exterior surface defines an opening for providing access to the interior cavity.

13. The urinal device of claim 11, wherein the bracket arm is spring-loaded.

14. The urinal device of claim 11, wherein the first actuator comprises hook member, the hook member configured to engage a grip portion of the waste collector assembly.

15. The urinal device of claim 11, wherein the waste collector assembly is configured to rotate around a pivot point as the waste collector assembly extends from the housing into an open configuration, and the waste collector assembly is configured to return to a closed configuration by rotating around the pivot point.

16. The urinal device of claim 11, wherein the bracket arm further comprises an extender wedge located on a surface of the bracket arm configured to facilitate returning the waste collector assembly from a folded configuration to a container configuration.

17. The urinal device of claim 11, wherein the bracket arm comprises two extender members configured to engage the waste collector.

18. The urinal device of claim 11, wherein a bottom surface of the waste collector bracket defines a channel, the channel being configured to engage a top surface of the bracket arm.

19. A method for using a urinal device comprising:
activating a urinal device from a closed configuration from an open configuration, wherein activating the open configuration comprises extending a waste collector assembly from an interior cavity of the urinal device;
providing a disposable substance to the waste collector assembly; and
detaching a portion of the waste collector assembly for disposal.

20. The method of claim 19, further comprising:
returning the urinal device from the open configuration to the closed configuration;
positioning another portion of the waste collector assembly in the interior cavity of the urinal device; and
reactivating the urinal device from the closed configuration to the open configuration.
